(19) United States
(12) Patent Application Publication
(10) Pub. No.: US 2002/0082467 A1
(43) Pub. Date: Jun. 27, 2002

(54) Prosthetic graft device with ventricular apex attachment apparatus

(76) Inventor: Louis Campbell, Austin, TX (US)

Correspondence Address: Timothy Scott, SulzerMedica USA Inc., Suite 1600, 3 Greenway Plaza, Houston, TX, 77046-0391, US

(21) Appl. No.: 09/745,398

(22) Filed: Dec. 21, 2000

(51) Int. Cl. A61N 1/362

(52) U.S. Cl. 600/16

(57) ABSTRACT

A prosthetic graft device for insertion through a body wall of an organ such as the heart includes a tubular graft and an annular, resilient element at a proximal end of the graft. The resilient element has an undeformed diameter greater than a principal diameter of the graft. Near the resilient element, but distal therefrom, a flange such as a sewing ring is coupled to an outer wall of the tubular graft. A cardiac assist device may be coupled to the tubular graft. Also, a method of securing a prosthetic graft device through a body wall including the step of folding a resilient annular ring attached to a tubular graft such that the resilient ring assumes a first, collapsed configuration having a cross-sectional area smaller than the cross-sectional area of the undeformed ring. The resilient ring is positioned through a body wall until a flange on an outer wall of the tubular graft contacts an outer surface of the wall. The ring is then allowed to resiliently deform to a second configuration, having a larger diameter then the first configuration, capturing the body wall between the resilient ring and the flange.

### FIELD OF THE INVENTION

The present invention relates to implantable prosthetic grafts and in particular to implantable prosthetic grafts or devices which may be attached through a cardiac wall into a chamber of the heart.

### BACKGROUND OF THE INVENTION

As a result of age or disease, the human heart may become weakened and unable to circulate sufficient blood to sustain the life or health of the patient. In certain circumstances, a cardiac assist device may be provided to increase blood flow volume and pressure. A cardiac assist device may comprise an axial-flow, non-pulsatile pump, or a left ventricular assist device (LVAD) of a type known in the art. Such a device may be connected to a chamber of the heart (typically the left ventricle via the left ventricular apex) by inserting a cannula or prosthetic graft through the heart wall without opening the heart beyond a necessary incision for insertion.

Insertion of a pump or LVAD through the wall of the heart is obviously a traumatic event for the heart wall, and attachment of such devices is frequently accompanied by increased bleeding and blood leakage at the attachment wound site. Healing of the attachment wound is made difficult by the repetitive, pulsatile pumping action of the left ventricular wall relative to the wall of the device or cannula. This difficult healing site notwithstanding, it is important that blood leakage around the graft be minimized and that areas of stagnation and clotting be reduced. The increased risk of blood loss associated with leakage at the wound site may present grave health risks to patients needing cardiac assist devices, since most such patients are already in poor health.

In addition to the heart, other organs and blood vessels may have a cannula or prosthetic graft inserted into the organ or blood vessel through a wall of the organ or vessel. The cannula may be used as an additional flow path, to inject fluids or drugs or to drain substances from the organ or vessel. In such cases it may also be desirable to have a prosthetic graft that can be inserted through the wall of the organ or blood vessel without a significant incision, and with minimal bleeding and/or leakage.

Vascular tubular prostheses may be inserted into the diseased portion of a blood vessel by surgically opening the vessel and suturing the prosthesis into position. However, it may be preferred to insert the prosthesis from a remote opening, such as the femoral artery, adjacent the groin, using a catheter system. Remote insertion eliminates the need to open a major body cavity and may diminish the potential surgical complications.

In cases of remote insertion of vascular grafts, it is generally desirable to insert the graft prosthesis, using a catheter, in a collapsed or compressed condition and then to expand the prosthesis when it has been moved from the remote location to the location to be repaired. One reason for this is that it is desirable to avoid substantially occluding the blood flow during the insertion process. By collapsing the prosthesis, the prosthesis may be readily positioned inside the vessel.

There are generally two techniques for expanding a collapsed prosthesis once it is in position at the location to be repaired. One technique uses an expandable metal prosthesis that is expandable by a mechanically supplied force. In a first, collapsed configuration, the prosthesis has a relatively smaller diameter, and in a second configuration, it has a radially expanded configuration, contacting and securing the prosthesis on either side of the diseased vessel wall. The prosthesis may be a malleable metal ring, toroid, or cylinder that may be expanded by a mechanical force from, for example, a balloon catheter to set the prosthesis in its expanded diameter, inside the neck portion, proximate to the diseased portion of the vessel.

The second technique for expanding a collapsed prosthesis is to use a self-expanding prosthesis, which may be compressed against a resilient biasing force. Once in position, the prosthesis is allowed to resiliently expand into contact with the vessel wall by removing the biasing force.

While a wide variety of methods have been proposed for the problem of effectively bypassing diseased tissue, these methods may not be effective for controlling bleeding associated with insertion of a prosthesis through the wall of a blood vessel or organ such as the heart. Thus, there is a continuing need for enhanced solutions to the problem of repairing diseased vessels and in general to the problem of effectively securing prosthetic devices through the internal walls of body passages or organs.

### SUMMARY OF THE INVENTION

According to one aspect of the present invention, a prosthetic graft device for insertion through a vessel or organ wall (such as a heart wall) includes a tubular graft and an annular, resilient element at a proximal end of the graft device. The resilient element has an expanded or undeformed diameter greater than a principal diameter of the graft. Near the resilient element, but distal therefrom, a flange is attached to an outer wall of the tubular graft. In one embodiment, the flange comprises a sewing ring.

According to another aspect of the present invention, a prosthesis for insertion through a body wall such as the wall of a heart includes an annular, resilient spring element and a tubular graft. A proximal end of the graft may advantageously be coupled to the spring element. The spring element has an expanded or undeformed diameter greater than a diameter of the graft. A flange on the graft is brought into contact with an outer surface of the body wall. The spring element is then allowed to expand against an inner surface of the body wall, mechanically capturing the body wall between the spring element on an inner surface of the body wall and the flange on an outer surface of the body wall.

In another aspect of the invention, a cardiac assist device has a tubular graft coupled to the assist device. The graft has a first annular, resilient ring coupled to a proximal end thereof and a second annular ring distal from said first resilient ring such that a body wall may be captured between the two rings. In one aspect of the invention, the body wall is a heart wall.

According to yet another aspect of the present invention, a method of securing a prosthetic graft through a body wall includes the step of folding a resilient annular ring attached to a tubular graft such that the resilient ring assumes a first (collapsed) configuration having a cross-sectional area smaller than the cross-sectional area of the undeformed ring. The resilient ring is inserted through a body wall until a flange on an outer wall of the tubular graft contacts an outer surface of the body wall. The resilient ring is then allowed to resiliently deform to a second (expanded) configuration, having a larger diameter than the first configuration, thereby capturing the body wall between the resilient ring and the flange.

The details of one or more embodiments of the invention are set forth in the accompanying drawings and the description below. Other features, objects and advantages of the invention will be apparent from the description of the invention, the drawings, and from the claims.

### BRIEF DESCRIPTION OF THE DRAWINGS

FIG. 1 is a view of a prosthetic graft device according to an embodiment of the present invention with attachment apparatus connected to a human heart.

FIG. 2 is a front view of the prosthetic graft device of FIG. 1.

FIG. 3 is generalized top plan view of a resilient, collapsible ring for use in the prosthetic graft device of FIG. 1 and 2.

FIG. 4 is a perspective view of the ring of FIG. 3 in a collapsed configuration.

FIG. 5 is a front elevational view of a prosthetic graft device according to an embodiment of the present invention, with a delivery apparatus coupled thereto.

FIG. 6 is a cross-sectional view taken generally along the line 6-6 in FIG. 5.

FIG. 7 is an enlarged, partially sectioned view of the delivery apparatus shown in FIG. 5.

FIG. 8 is a cross-sectional view taken along line 8-8 in FIG. 7.

FIG. 9 is a cross-sectional view taken along line 9-9 in FIG. 7.

FIG. 10 is an enlarged front elevational view of a prosthetic graft device according to an embodiment of the present invention, maintained in a collapsed position by a retention loop.

FIG. 11 is a front elevational view of a portion of the retention loop of FIG. 10.

FIG. 12 is a front elevational view of another embodiment of a prosthetic graft device according to the present invention and an insertion device therefor.

FIG. 13 is an enlarged view of the prosthetic graft device shown in FIG. 12.

FIG. 14 is a top view of the prosthetic graft device of FIG. 10 prior to release from a catheter.

FIG. 15 is a top view of the embodiment of FIG. 12 prior to release from a catheter.

FIG. 16 is a view of the embodiment of FIG. 12 coupled to a human heart.

### DESCRIPTION OF PREFERRED EMBODIMENTS

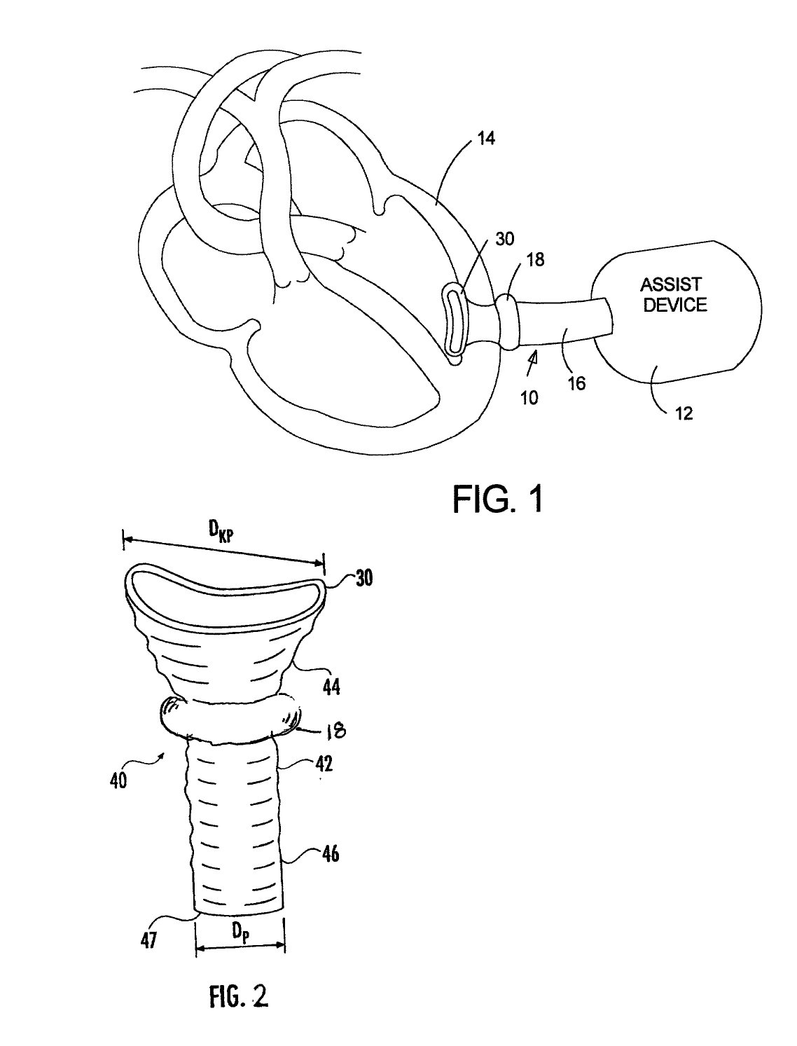

Referring to the drawing wherein like reference characters are used for like parts throughout the several views, a prosthetic graft device 10 connected to a cardiac assist device 12 is shown in FIG. 1. The prosthetic graft device 10 is connected to the heart 14 of a patient by insertion through an incision in the wall of a chamber of the heart. Insertion of the graft device 10 into the ventricles of the heart is illustrated, but the graft device may be inserted into any chamber of the heart or through the wall of a vein or artery or other organ, wherever access to the interior of a body organ or vessel is needed through a wall and it is not desirable to access the interior of the blood vessel or organ to attach the graft device. The graft device 10 has an expandable ring 30 at a proximal end of the graft. The ring 30 is resilient and can be collapsed to facilitate insertion of the graft through the wall of the organ, for example, through the wall of the heart. A flange 18 is coupled to the graft device 10 distally from the proximal expandable ring. In a preferred embodiment, the flange comprises a sewing ring. The flange 18 comes into contact with the outside surface of the heart wall as the proximal ring 30 expands against the interior wall of the heart. Both the flange 18 and the expandable ring 30, therefore, contact adjacent wall surfaces and capture the wall therebetween. An effective seal is formed without surgical intervention into the interior of the chamber, e.g., the left ventricle. The flange may be sutured to the outside of the heart wall to more fully secure the prosthetic device to the heart wall.

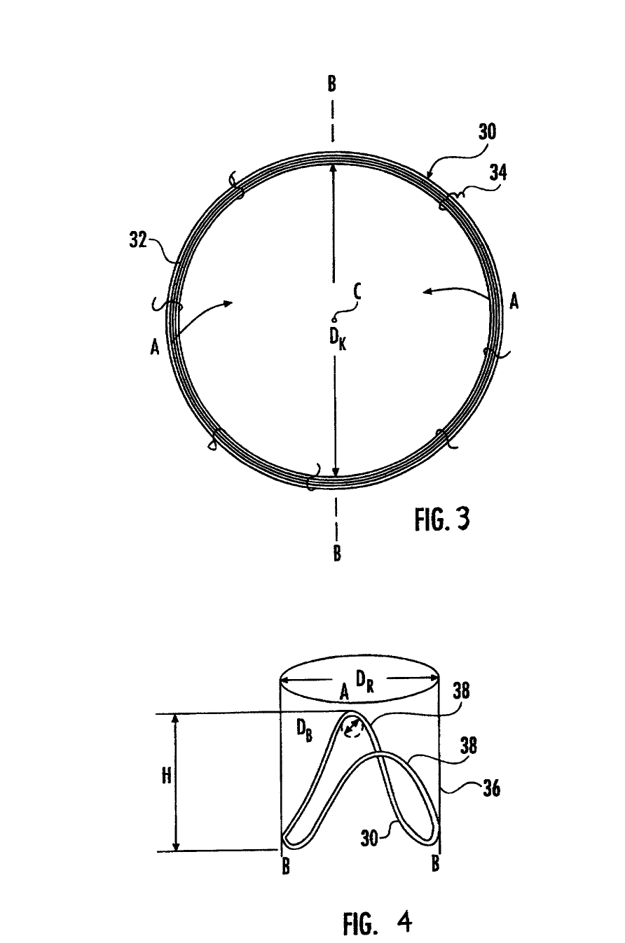

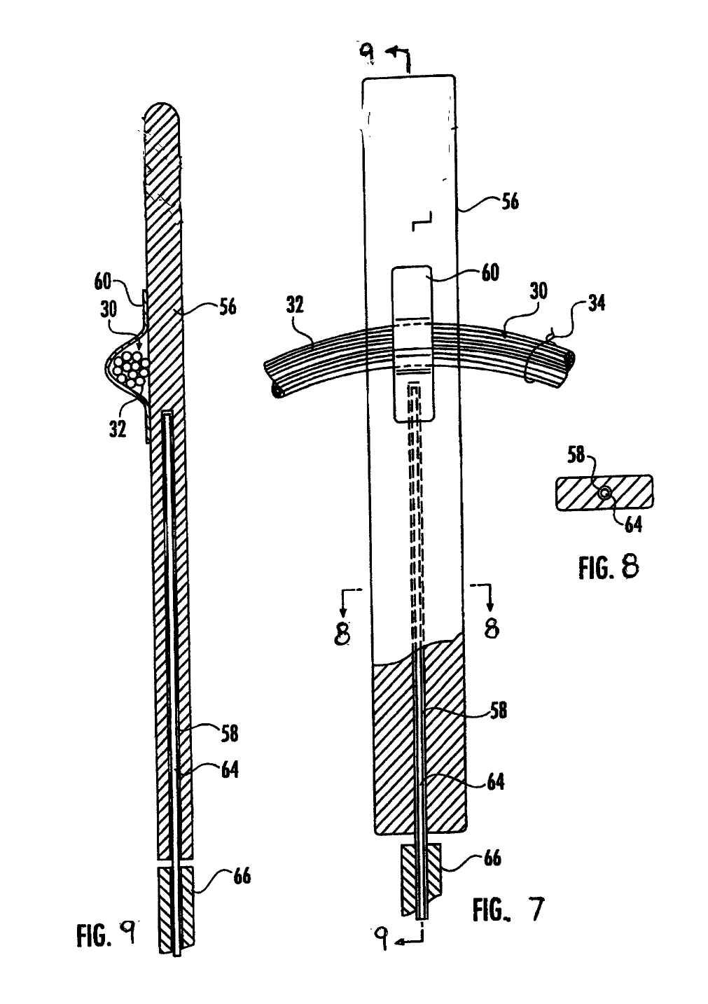

An annular, resilient clamping ring 30 may be formed of a plurality of strands 32 of resilient wire as shown in FIGS. 3, 7 and 9. One embodiment of the ring 30 may be formed by wrapping a single length of wire around a mandrel (not shown) having a central axis “C” and then securing the strands into a bundle using ties 34. The ties 34 may be formed from surgical suture material. Of course, the ring 30 may be formed by a variety of other techniques including the use of a single strand of wire, the use of multiple strands of helically intertwined wire, as in multi-strand wire rope, or any other suitable technique which forms a highly resilient annular ring.

The number of coils or strands 32 can be varied according to the wire utilized and the particular application involved. However, in one embodiment, the number of strands 32 utilized is approximately 8 to 12 as shown in FIG. 9. However, the number of coils or strands 32 may vary from as few as 2 to as many as 100 or possibly more.

While a variety of different wire diameters may be utilized, the individual strands 32 may have a diameter of from about 0.05 to 1 mm. In one embodiment a wire strand 32 may have a diameter of about 0.1 mm. The strands 32 may be made of any highly resilient metal or plastic material, including a nickel titanium alloy such as Nitinol. Generally the super-elastic or stress-induced martensitic form of Nitinol is preferred, although other biologically compatible metals or alloys, such as shape memory Nitinol or stainless steel, may also be used.

Referring to FIGS. 3 and 4, the ring 32, before compression, may have a diameter, D_{K}, which is considerably greater than the diameter of an opening or incision 36 in the wall of the organ or vessel through which the graft device is to be inserted. As indicated in FIG. 4, two diametrically opposed points “A” on the undeformed ring 30 may be deflected towards one another. As indicated by the arrows, this causes the ring 30 to fold along its diametric axis “B”. In this configuration, the ring 30 may be inserted into the incision 36 in a configuration having a reduced diameter D_{R}.

As a result of the folding along the diametric axis “B,” the loops 38, which include the folded tips “A,” extend proximally relative to the points “B” which are along the diametric axis of folding. Because the device is suitable for use in cardiac surgery, as used herein, the term “proximal” refers to a direction toward and through a wall of a body organ (e.g., the heart) or vessel and the term “distal” refers to the direction away from the organ or vessel, that is, in a direction towards an attending physician who might be manipulating the graft device. The proximal end of the graft device, with the ring 30, is inserted through the wall of the organ or vessel until the flange 18 contacts an outer surface of the wall. Once in position inside the body wall, the ring 30 opens to an expanded diameter and makes continuous contact with the internal vessel wall.

The smallest permissible bending diameter without plastic deformation, D_{B}, shown in FIG. 4, depends on the material, the thickness of the ring 30 and the individual strands 32 which may make up the ring 30. According to Hooke's law, the strands 32 can be regarded as parallel connected springs whose deflection characteristic values are additive and whose individual low radial tension forces add up to a total tension force which depends on the number of strands 32. When the entire ring 30 is compressed, each individual strand 32 has a bending diameter approximately corresponding to the minimum bending diameter D_{B} of the individual strand 32. As an approximation, the minimum bending diameter D_{B} is approximately ten times the wire diameter. This suggests that the ring wire diameter should be kept low. However, the ring's expansion force, which helps to ensure effective sealing on the inner organ wall, is a function of its diameter, suggesting conversely that the wire diameter be increased. This tradeoff between collapsibility for ease of insertion and expansion force for effective sealing can be optimized by using a plurality of strands 32, whose diameter controls the minimum bending diameter, to form a bundle whose composite diameter controls the expansion force. Thus a ring 30 with a high expansion force can be shaped to a relatively small compressed configuration. After being released from a catheter having, for example, a conventional diameter of from 4 to 6 mm, the ring 30 may return to its original shape.

A prosthetic device 40 may include an annular ring 30 and a graft 42, as shown in FIG. 2. The graft 42 may be generally tubular and made of a fabric or film secured on one end to the ring 30. The graft 42 may have a diameter D_{P} that is smaller than the diameter D_{K} of the ring 30. Due to the connection between the ring 30 and the end of the graft 42, there is a diameter D_{KP} at the junction point between the ring 30 and the graft 42. The ring 30 may expand the end of the tubular graft 42 to a stop or deformation limit, after which no further expansion occurs. Thus, the ring 30 may expand the graft 42 in the region proximate to the ring 30 so that the diameter of the graft 42 gradually tapers in the region 44 down to a relatively constant diameter region 46, terminating in a free end 47. Alternatively, the graft 42 could be preformed in the flared shape shown in FIG. 2.

Any of a variety of fabric materials compatible with human implantation may be utilized to form the graft 42. For example, the graft 42 may be formed of flexible woven or knitted textiles made of Dacron, Teflon, or other materials. It is advantageous if the tubular graft 42 is made of a material, which does not change its circumference readily. The ring 30 can be connected with the region 44 by means of sutures or bonding. In one embodiment, the graft material is pulled over and around the periphery of ring 30, then folded back inside the tubular capturing ring 30 within a toroidal loop of the graft material, which is secured by sutures of other fixation means, e.g. staples. Thus, it may be advantageous that the diameter D_{K} of the ring 30 be considerably greater than the diameter of the portion 46 of the graft.

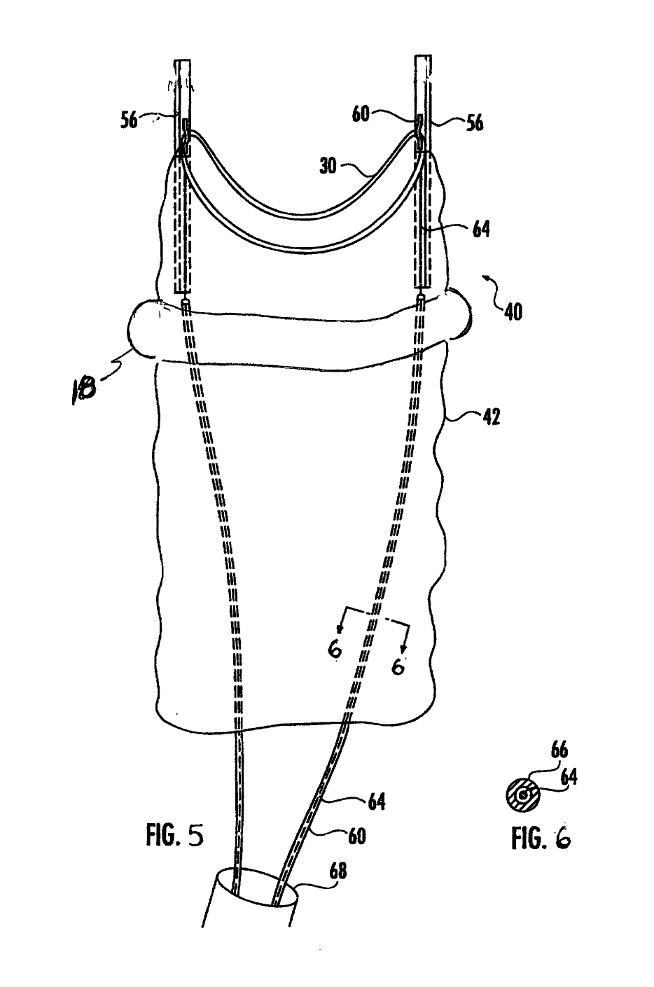

Turning now to a method for positioning the prosthetic device 40 in a desired location within a body organ, a retention device 56, shown in FIG. 5, may be secured to the ring 30 on at least two diametrically opposed orientations so that the device 56 extends generally parallel to the axis of the prosthetic device 40. The device 56 may include a passage 58 in one end and a bracket 60 that secures the device 56 to the ring 30. Alternatively the passage 58 may be replaced by wire restraining brackets (not shown). The device 56 may be engaged by a wire 64 which extends into the passage 58 and by a tube 66 which encircles the wire 64, as indicated in FIG. 6. Advantageously, the device 56 and the tube 66 are made of sufficiently rigid material that pushing against the device 56 by the wire 64 or the tube 66 results in displacement of the prosthetic device 40 through the incision 36. The wire 64 may have a diameter of about 0.3 to 1 mm.

The prosthetic device 40 may be compressed to fit into a tubular catheter 68, for transferring the prosthesis from a remote entry point to the repair site. The catheter 68 may be inserted into an incision in the body, and moved through a body cavity such as a blood vessel to a position at the wall of a ventricle of the heart, for example, where one may wish to position the annular ring 30. Once in position, the prosthetic device 40 may be pushed out of the catheter 68 using the tubes 66.

More particularly, the tubes 66 are extended into the body from the exterior thereof by the surgeon while maintaining the catheter 68 in a fixed position so that the prosthetic device 40 is placed in a desired position as the catheter 68 is backed away. If desired, the brackets 60 may be made of X-ray opaque material such as platinum, iridium or gold to serve as an X-ray marker.

While the above-described procedure for placing the prosthetic device 40 may be useful in some applications, it is desirable to further facilitate accurate and controllable placement of the prosthetic device 40 in a particular location. Once the ring 30 is allowed to expand against the inner surface of the body wall, any re-positioning must be done against the resistant force of the ring 30. Thus, it is advantageous to continue to confine the ring 30 after the prosthetic device 40 is removed from catheter 68, until the prosthesis 40 is accurately positioned.

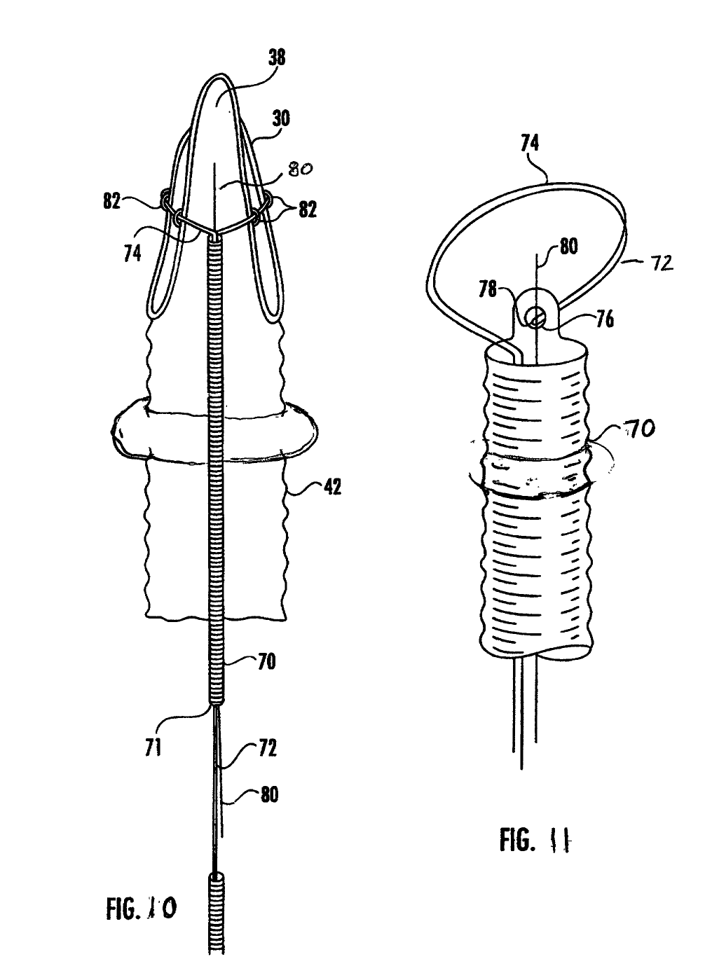

Once the prosthetic device 40 is positioned as desired, ring 30 may be allowed to expand by removing the constraint. To this end, a Bowden tube 70 telescopically retains a wire loop 72, as shown in FIGS. 10 and 11. The loop 72 extends axially through the tube 70, forms an annular ring 74 and passes through a hole 76 in the proximal free end of the Bowden tube 70. At this point, the looped end 78 of the wire loop 72 receives a blocking wire 80, where the looped end 78 extends out of the hole 76. Referring to FIG. 10, the Bowden tube 70 extends along the exterior of the prosthetic device 40 to a point distal to the loops 38 of collapsed ring 30. The annular ring 74 of loop 72 extends around the periphery of the loops 38 at a relatively central location along their length and through eyelets 82 secured to the ring 30.

Because the collapsed ring 30 presses outwardly against the annular ring 74, there is a force tending to draw the looped end 78 back through hole 76, thereby releasing collapsed ring 30. To prevent this, blocking wire 80 is captured between looped end 78 of wire loop 72 and Bowden tube 70 adjacent the hole 76 in the proximal free end of the Bowden tube 70. Pulling on a distal end of the blocking wire 80 is necessary to overcome the friction holding the blocking wire in place. In addition, the blocking wire 80 may be permitted to extend a relatively substantial distance beyond the proximal free end of the Bowden tube, as shown in FIG. 10, although it should not extend past collapsed ring 30.

In this way, the blocking wire 80 may be held in place until withdrawn axially, releasing looped end 78 so that the wire loop 72 may be withdrawn, thereby releasing the collapsed ring 30 and allowing it to spring open at a desired location. The blocking wire 80 may extend, inside the Bowden tube 70, to the distal end of the Bowden tube or may exit the tube through a gap 71 in the tube, as shown in FIG. 10.

Referring to FIG. 14, the catheter 68 encircles the prosthetic device 40 that in turn encircles a pair of tubes 66 with wires 64 extending through them. If necessary, a guide wire 104 may be included which may be used initially to guide the catheter to the desired location and to maintain a path for returning to the same location with additional elements, if necessary. The Bowden tube 70 with the looped wires 72 and blocking wire 80 also extends inside the catheter 68 between the catheter and the prosthetic device 40.

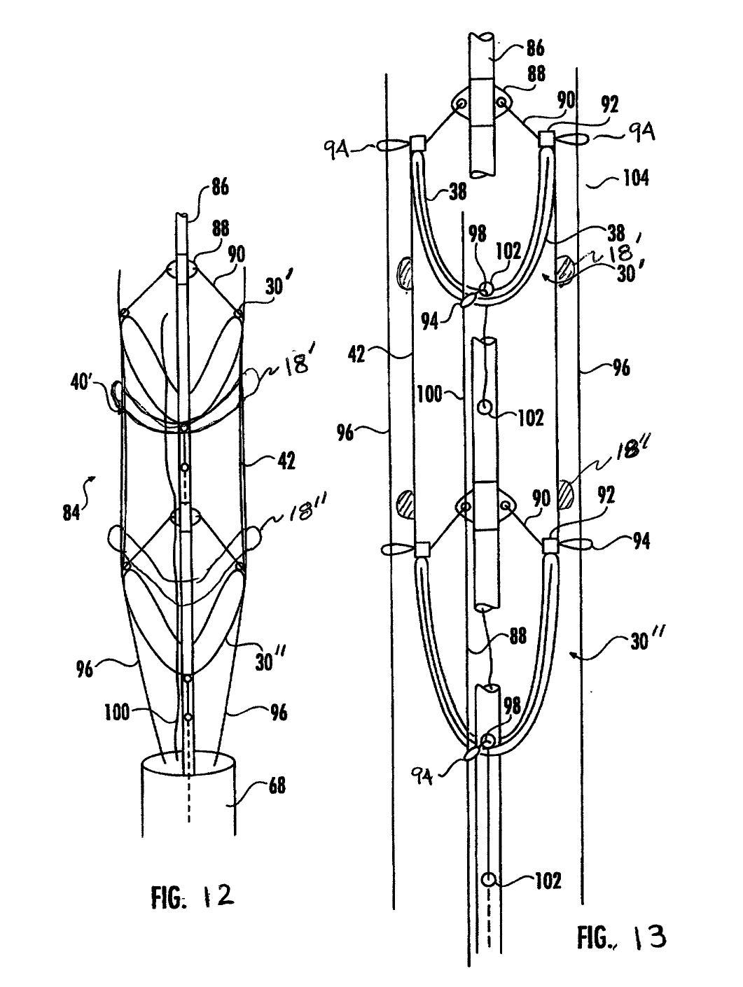

In still another embodiment, a retaining mechanism 84, shown in FIGS. 12 and 13, retains the prosthesis in a compressed configuration to accurately locate it at the desired position within a passage. The mechanism 84 may control a prosthetic device 40′ having a pair of rings 30′ and 30″, connected by a graft 42, in a compressed position inside a catheter 68. A first flange 18′ is adjacent a first ring 30″ and a second flange 18″ is adjacent a second ring 30″. A guide wire catheter 86 extends axially through the prosthetic device 40′. A plurality of ringlets 88 extends off of the catheter 86. Each of the ringlets 88 connects to wire loops 90 that in turn connect to eyelets 92 at the free ends of the loops 38. Referring to FIG. 13, each of the wire loops 90 slidably and releasably extends through the eyelet 92 and forms a loop end 94. A blocking wire 96 extends through the loop ends 94. A portion of each of rings 30′ and 30″ along its folding axis “B” (see FIG. 3 or FIG. 4) is wrapped by a wire loop 98 which is engaged through a loop end 94 on its free end by blocking wire 100. The wire loop 98 may wrap around and over the rings 30′, 30″, over the outside of the guide wire catheter 86 and into the interior of the catheter 86 through an opening 102. Each of the rings 30′ and 30″ on opposed ends of the graft 42 includes the same parts and may be operated in the same way.

Thus, to adjust the extent of folding or the proximal-distal height of the rings 30′, 30″ in the orientation shown in FIG. 13, it is simply necessary to pull outwardly on the wires 98 which may be connected together to a single wire 103 that extends to the exterior of the patient. To decrease the height and to decrease the compression of the rings 30′, 30″, the tension on the wire loop 98 may be relaxed, allowing the natural spring forces of the rings 30′, 30″ to cause the bending of the ring 30′, 30″ to be relieved and the ring height to be reduced.

After the catheter 68 is positioned in the desired location, the assembly may be ejected from the catheter using the techniques described previously. The amount of compression of the rings 30′, 30″ may be adjusted so that the apparatus 84 can be temporarily positioned at a desired location. If it is determined that the location is not precisely correct, the apparatus can be re-compressed, by operating the loops 98, to allow repositioning of the apparatus 84 to a new location. In this way, it is possible to selectively adjust the position of the prosthetic device 40′, even after the prosthesis has previously been released within the body organ or vessel. If an error is initially made, it is easy to reposition the prosthesis, as necessary. Once the prosthetic device is located at the desired location, the blocking wires 100 and 96 can simply be pulled out of the assembly through the catheter 68. This allows the prosthetic device 40′ to expand, irreversibly. The catheter 86 may be removed thereafter.

If desired, each of the loops 98 can be connected 20 by an independent wire to the exterior of the patient or, as described previously, the wires 98 may be connected so that only one single wire extends outwardly.

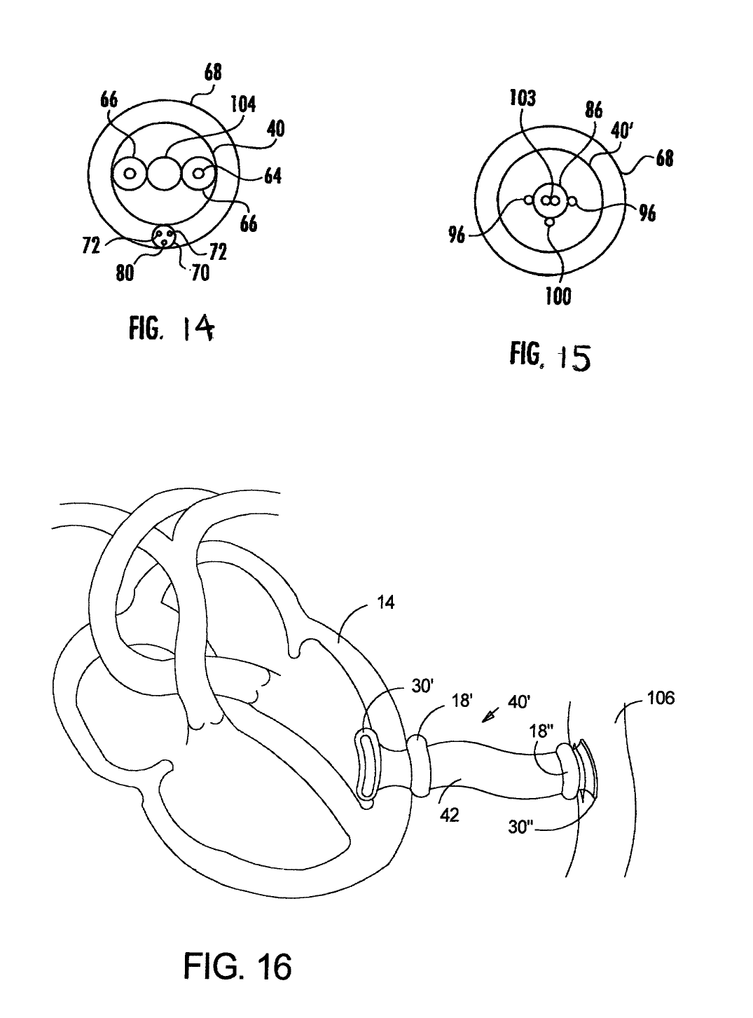

Referring now to FIG. 15, illustrating the catheter bundle for the embodiment illustrated in FIGS. 12 and 13 prior to release from the catheter 68, the catheter 68 encircles the prosthetic device 40′. In the interior of the prosthetic device 40′ is the guide wire catheter 86, with one or more of wires 103 that may be used to control the position of the folded portion of the annular rings 30′, 30″. Outside of the guide wire catheter 86 are a pair of wires corresponding to the blocking wires 96 and 100.

The apparatus 84 with two annular rings 30, 30″ may be particularly useful in connecting a chamber of the heart directly to a blood vessel. As illustrated in FIG. 16, the first ring 30′ of the apparatus 84 may be placed within the left ventricle of the heart 14, for example. The first flange 18′ would rest against the outer wall of the heart. The second ring 30″ would be inserted into an artery 106, bypassing the mitral valve and the left atrium. The second flange 18″ would rest against an outer wall of the artery. The apparatus 84 could be inserted into the desired location in the body by passing the apparatus through the artery or blood vessel, for example, through the femoral artery.

While the present invention has been described with respect to a limited number of preferred embodiments, those skilled in the art will appreciate numerous modifications and variations therefrom. For example, while the device has been described in some instances as a vascular stent for treating aneurysms, the invention may be applicable to securing any device to an internal passage. In addition, it should be appreciated that certain embodiments of the present invention may have only one or more of the advantages described above or may instead have other advantages not specifically mentioned herein. It is intended that the appended claims cover all such modifications and variations as fall within the true spirit and scope of the appended claims.

What is claimed is:

1. A prosthetic graft device for insertion through a wall of a body vessel, the graft device comprising
a tubular graft having a proximal end and a distal end, said tubular graft having a first diameter,
a first resilient annular ring coupled to said tubular segment at said proximal end, said first resilient annular ring having a second, collapsed diameter and a third, expanded diameter greater than said first diameter, and
an annular flange attached to said tubular segment near said distal end of said prosthesis and spaced away from said first resilient annular ring.

2. The prosthetic graft device of claim 1 wherein said first resilient annular ring comprises a plurality of strands.

3. The prosthetic graft device of claim 1 further comprising a second annular ring spaced proximally from said first resilient annular ring, said second annular ring having a fourth, expanded diameter less than said third, expanded diameter and greater than said first diameter.

4. The prosthetic graft device of claim 3 further comprising a plurality of resilient annular rings other than said first annular ring, each succeeding ring of said plurality of resilient annular rings having an expanded diameter equal to or less than the expanded diameter of an immediately preceding ring.

5. The prosthetic graft device of claim 4 wherein said first resilient annular ring and a ring immediately adjacent said first resilient annular ring have substantially equal extended diameters.

6. The prosthetic graft device of claim 1, said flange further comprising a diameter less than said third, expanded diameter.

7. A cardiac assist device comprising
a blood pump having a blood intake port and a blood exhaust port, and
a vascular prosthesis coupled to said blood exhaust port, said vascular prosthesis having
a distal end adjacent said blood pump and a proximal end remote from said blood pump,
said prosthesis comprising
a tubular segment extending from said proximal end to said distal end, said tubular segment having a first diameter,
a first resilient annular ring coupled to said tubular segment at said proximal end, said first resilient annular ring having a second, collapsed diameter and a third, expanded diameter greater than said first diameter, and
an annular flange coupled to said tubular segment near said distal end of said prosthesis and spaced away from said first ring.

8. The cardiac assist device of claim 7 wherein said first resilient annular ring comprises a plurality of strands.

9. The cardiac assist device of claim 7 further comprising a second resilient annular ring spaced distally from said first annular ring, said second annular ring having a fourth, expanded diameter less than said third, expanded diameter and greater than said first diameter.

10. The cardiac assist device of claim 9 further comprising a plurality of resilient annular rings other than said first annular ring, each succeeding ring of said plurality of resilient annular rings having an expanded diameter equal to or less than the expanded diameter of an immediately preceding ring.

11. The cardiac assist device of claim 10 wherein said first resilient annular ring and a ring immediately adjacent said first resilient annular ring have substantially equal extended diameters.

12. The cardiac assist device of claim 7, said flange further comprising a diameter less than said third, expanded diameter.
